(19) United States
(12) Patent Application Publication
(10) Pub. No.: US 2008/0254153 A1
(43) Pub. Date: Oct. 16, 2008

(54) Water Dispersible Composition and Method for Preparing Same

(76) Inventors: Junkuan Wang, Lonay (CH); Raymond Bertholet, Blonay (CH); Pierre Ducret, St-Saphorin-Sur-Morges (CH)

Correspondence Address: WINSTON & STRAWN LLP;PATENT DEPARTMENT, 1700 K STREET, N.W., WASHINGTON, DC, 20006, US

(21) Appl. No.: 12/067,417

(22) PCT Filed: Sep. 19, 2006
(86) PCT No.: PCT/EP2006/066511

§ 371 Date: Mar. 19, 2008

(30) Foreign Application Priority Data
Sep. 20, 2005 (JP) 05020453.6

(51) Int. Cl. A61K 36/82; A61K 36/00; A61K 31/215; A61K 31/70; A61K 31/05; A61K 31/01; A61K 31/12; A61K 31/045; A61K 31/352; A61K 31/7048; A61K 36/45; A61K 36/73; A61K 36/87; A61K 36/736; A61K 36/752; A61K 36/28; A61K 36/48; A61K 36/31; A23L 1/221; A23L 1/30; A61P 3/00; A61P 35/00; A61P 9/00; A61P 37/00; A61P 27/02; A61P 17/00; A61K 36/899; A61K 36/8962

(52) U.S. Cl. 424/729; 424/725; 514/552; 514/23; 514/731; 514/762; 514/691; 514/729; 514/456; 514/27; 424/732; 424/765; 424/766; 424/735; 424/736; 424/764; 424/757; 424/755; 424/754; 424/750; 426/442; 426/384; 426/655; 426/648

(57) ABSTRACT

The invention pertains to a water dispersible, preferably a powder composition of water extractible bioactive components of exclusively fruit or vegetable or plant origin. These components are obtainable by: a) subjecting a selected fruit or vegetable or plant material to homogenisation in weak alkaline conditions and at moderate temperature; b) separating the liquid extract from the homogenised mass and subsequently bringing it to neutrality; and c) eventually concentrating or drying, preferably by freeze drying, the neutralized liquid extract. The composition can be used as primary composition in the preparation of a food product for oral administration, a food supplement, a pet food product, a pet food supplement, a cosmetic preparation or a pharmaceutical preparation.

### FIELD OF THE INVENTION

The invention relates to a water dispersible composition, preferably a powder composition consisting of water extractible bioactive components of exclusively fruit or vegetable or plant origin, its method of preparation as well as its use as primary composition in the preparation of various food products, food supplements, pet food products, pet food supplements or cosmetic or pharmaceutical preparations.

### BACKGROUND OF THE INVENTION

Pigments and bioactive compounds extracted from fruits or plant materials are widely used in the food industry as functional ingredients. Among all of them, wolfberry (Lycium barbarum) is one of the most valued functional ingredients in China, especially for its benefits for eyesight, the immune system, and its anti-ageing properties, associated with the multiple bioactive compounds present in the fruit. It is traditionally consumed through hot water extraction.

Many other bioactive ingredients are also well perceived by consumers for their beneficial properties, but their application in e.g. food products is either difficult or provides poor bioavailability. In addition to that, current extraction techniques afford the desired ingredients in very low yield and lead frequently to some deterioration of the genuine properties of the extracted ingredients. As a matter of fact, fruits are usually rich in reducing sugars, making the drying and handling of their powder very difficult.

A large number of extraction techniques are already known. For example, in WO03020053, a process for extracting carotenoids from carotenoid-containing plant matter is described. It comprises (i) mixing the plant matter with water to achieve Brix not greater than 10 DEG; (ii) crushing the mixture from step (i) and separating the solids from the liquid to obtain two phases, i.e. pulp and serum; (iii) extracting the pulp to obtain carotenoid-containing plant oleoresin. Such a water extraction technique is natural and preserves to some extent the properties of the carotenoid extract, but is not as efficient as compared to the use of a solvent.

U.S. Pat. No. 6,648,564 describes a process for forming, isolating and purifying xanthophyll crystals by saponification of a xanthophyll diester-containing plant extract in a composition of propylene glycol and aqueous alkali to form xanthophyll crystals. The substantially pure xanthophyll crystals so obtained are suitable for human consumption and can be used as a nutritional supplement and as an additive in food. However, solvent extraction techniques are more difficult to handle, and using solvent definitely impairs the genuine natural character, its perception from the consumer and/or some of the nutritional functions of the product.

WO 2005/020948 discloses a method for extracting carotenoids from fruits or vegetables relatively rich in carotenoids like e.g. lycopene or carotenes by subjecting the fruit or vegetable material to boiling with water at neutral pH, separating the liquid phase and eventually drying it to afford a fairly lycopene rich material. Said material is unfortunately hard to get in its dry form and consequently of limited use.

Conventional extraction techniques usually extract a few compounds of the plant or fruit material, leaving some other bioactive compounds in the rest. For example, polysaccharides, polyphenols and other non lipophilic compounds are not extracted together with the lipophilic components such as carotenoids, lipophilic vitamins and other lipids.

It is thus an object of the present invention to address the above problems by providing a method for extracting and collecting bioactive ingredient from a fruit or a plant material with improved stability, miscibility, dispersibility in aqueous systems and, also, with enhanced bioavailability as compared to similar extracts from the prior art. Further objects of the invention shall become apparent from the details of the description here below.

### SUMMARY OF THE INVENTION

As a first object the invention provides a process for preparing a primary water dispersible composition consisting of water extractible lipophilic and non lipophilic bioactive components of exclusively fruit or vegetable or plant origin, which comprises:

a) subjecting a selected fruit or vegetable or plant material to homogenisation at a pH of about 8 to 11 and at a temperature comprised between about 20-25 and 50° C.,

b) separating the liquid extract from the homogenised mass and subsequently bringing it to neutrality, and

c) eventually concentrating or drying the neutralized liquid extract.

Another object of the invention is a primary composition consisting of water extractible lipophilic and non lipophilic bioactive components of exclusively fruit or vegetable or plant origin as well, wherein the profile of said bioactive components is similar if not identical to that of the corresponding whole fruit or vegetable, and its use in the preparation of a food product for oral administration, a food supplement, a pet food product, a pet food supplement or a cosmetic or a pharmaceutical preparation.

Still another object of the invention is the use of the said primary composition for preparing an oral, cosmetic or pharmaceutical composition intended for improving skin health, in particular for photo protection of the skin or for protecting skin tissue against ageing or for preparing an oral, topical or pharmaceutical composition intended for eye health or an oral, cosmetic or pharmaceutical composition intended for stimulating the immune system or for preparing an oral, cosmetic or pharmaceutical composition for preventing or treating cardiovascular diseases or disorders or cancers or diabetes.

### DETAILED DESCRIPTION OF THE INVENTION

Within the following description, the term bioactive compound is understood to mean molecules or components showing biological activity or health impact when orally ingested or applied in cosmetics.

According to the invention the whole fruit or vegetable or plant material is subjected to homogenization in relatively weak alkaline conditions, i.e. at a pH of about 8 and higher, generally comprised between about 8 and 11. Said homogenization is carried out at a moderate temperature, most frequently at a temperature comprised between room temperature (usually 20 to 25° C.) and max. 50° C. so that to preserve as much as possible the integrity of all the bioactive ingredients which are present in the whole fruit or vegetable mass. More elevated temperatures, e.g. 60 or 70° C. or even higher can also be taken into consideration.

Depending on the fruits or vegetable or plant material subjected to treatment the latter material can be first washed with water; said washing is preferably carried out at room temperature (usually 20 to 25° C.) or slightly above. Doing so enables discarding highly soluble fruits or vegetable components like e.g. reducing sugars or highly hydrophilic components which provide undesired hygroscopy to the final extracts. Furthermore, discarding said highly soluble components consequently increases the concentration of bioactive lipophilic material.

Homogenization is performed according to techniques usual in the art, e.g. by means of high speed mixers or rotating mills to achieve the necessary crushing of all the fibrous and cellular material.

Insoluble, solid particles of the homogenized fruit or vegetable or plant mass are then separated from the liquid phase by means of any suitable technique, most conveniently by means of centrifugation. The resulting liquid phase, indeed a fairly clear suspension of coloured material in most instances, is subsequently brought to neutrality (pH 7), preferably by the addition the requested amount of a food grade acid.

Concentration of the neutralized liquid material is performed using any technique which will preserve the integrity of the extracted material, i.e. which will not or almost not impair the genuine properties of all the bioactive components initially present in the whole fruit or vegetable or plant material.

It is a preferred embodiment of the invention to dry the said liquid extract. For doing so, although spray drying may be considered in some instances, freeze drying is the preferred drying method. The powder composition so achieved is stable and easy to handle.

It has been observed that such extracts of exclusively fruit or vegetable or plant origin present an improved or increased bioactivity as compared to similar extracts prepared according to the prior art, definitely much closer to the activity which characterises said ingredients in their natural environment. Such extracts, i.e. preferably powder compositions of the invention comprise what is defined as “water extractible components” within the frame of the invention, i.e. mainly water soluble components such as polysaccharides, proteins or peptides, antioxidants like polyphenols and vitamins, minerals and also lipophilic components like e.g. carotenoids either entrapped in polysaccharides (e.g. starch derivatives, dextrin . . . ) or as protein complexes or associations.

The fruit or vegetable or plant material used within the frame of the invention can be in the form of vegetables, leaves, flowers, fruits, seeds and other parts of the plant, or a mixture thereof.

In a preferred embodiment, berries or any other flavonoid-, polyphenols- or carotenoid-rich fruit or vegetable or seeds are selected. For example, berries such as wolfberry, blueberry, cranberry, mulberry, blackberry, gooseberry, white currant, blackcurrant, red currant, raspberry, sea buckthorn, strawberry, arbutus berry or grapes and other fruits such as apples, melons, kiwi, cherries, red date, prunes, peaches, persimmons, citrus fruits such as mandarin, orange, tangerine, grapefruit, for example, may be used. Flowers such as chamomile, chrysanthemum, bitter orange, honeysuckle, jasmine and safflower may be used. Vegetables such as tomato, spinach, celery, carrots, pea, kale, parsley, watercress, cabbage, broccoli, lettuce, Brussels sprouts, collard greens, turnip greens, fennel or onions can also be used as well as seeds such as corn, black rice, cocoa, coffee and ingredients such as tea, thyme, sweet red pepper.

Fruits, vegetables or plant materials may be used in the form of fresh, concentrated or dried material, for example, air or freeze dried material.

The essential bioactive components of fruit, vegetable or plant material may comprise lipids, alkaloids, proteins, carbohydrates, carotenoids, polyphenolic compounds such as flavonoids, and vitamins or minerals, for example. In particular, the bioactive compounds may be flavonoids such as flavones (e.g. epigenin, luteolin or diosmetin), flavonols (e.g. quercetin, myricetin, kaempferol), flavanones (e.g. naringenin, hesperidin), catechins (e.g. epicatechin, gallocatechin), anthocyanidins (e.g. pelargonidin, malvidin, cyanidin) or isoflavones (e.g. genistein, daidzein); carotenoids such as carotenes and xanthophylls (e.g. lycopene, carotene, phytofluene, phytoene, canthaxanthin, astaxanthin, beta-cryptoxanthin, capsanthin, lutein, zeaxanthin, or those in the form of fatty acid esters; carbohydrates such as arabinogalactan proteins (e.g. lycium barbarum polysaccharide); vitamins (e.g. vitamin C, B, E . . . ); minerals (e.g. selenium, calcium, magnesium, potassium).

As referred to here above, the present invention provides a composition, preferably a powder composition having a similar profile of its essential nutrients similar if not identical to that the whole fruit or vegetable; furthermore it exhibits a good stability, miscibility with water and bioavailability. The powder compositions of the invention are indeed highly dispersible in an aqueous system, either in cold or hot water.

According to another embodiment of the invention such a composition is used as primary composition in the preparation of a food product for oral administration, a food supplement, a pet food product, a pet food supplement, a cosmetic or a pharmaceutical preparation.

Within that context, the said primary composition further comprises one or more of emulsifiers, stabilizers, antioxidants and other additives. Use is made of emulsifiers compatible in food, such as phospholipids, for example lecithin, polyoxyethylene sorbitan mono- or tristearate, monolaurate, monopalmitate, mono- or trioleate, a mono- or diglyceride. Use may also be made of any type of stabilizer that is known in food, in cosmetics or in pharmaceuticals. Use is made of any type of antioxidants that is known in food, in cosmetics or in pharmaceuticals. Use is made, as additives, of flavorings, colorants and any other additive known in food, in cosmetics or in pharmaceuticals. These emulsifiers, stabilizers, antioxidants and additives are added according to the final use of the primary composition.

The said primary composition may also contain synthetic or natural bioactive ingredients such as amino acids, fatty acids, vitamins, minerals, carotenoids, polyphenols, etc. that can be added either by dry or by wet mixing to said composition before pasteurization and/or drying.

According to a further aspect, the present invention relates to an oral composition comprising the primary composition described above in a foodstuff, in a food supplement, in a pet food product, in a cosmetic preparation or in a pharmaceutical preparation.

In a preferred embodiment, a food composition for human consumption is supplemented by the above primary composition. This composition may be a nutritional complete formula, a dairy product, a chilled or shelf stable beverage, a mineral or purified water, a liquid drink, a soup, a dietary supplement, a meal replacement, a nutritional bar, a confectionery, a milk or a fermented milk product, a yoghurt, a milk based powder, an enteral nutrition product, an infant formula, an infant nutritional product, a cereal product or a fermented cereal based product, an ice-cream, a chocolate, coffee, a culinary product such as mayonnaise, tomato puree or salad dressings or a pet food.

In this case, the primary composition can be dispersed in the above-mentioned foods or drinks so as to have a daily intake in bioactive nutrients as described above, which depends mainly on the fruit, vegetable or plant material utilized or on the desired effect and target tissue. The amount of the primary composition or food composition to be consumed by the individual to obtain a beneficial effect will also depend upon its size, its type, and its age.

The nutritional supplement for oral administration may be in capsules, gelatine capsules, soft capsules, tablets, sugar-coated tablets, pills, pastes or pastilles, gums, or drinkable solutions or emulsions, syrup or a gel, with a dose of about 0.1 to 100% of the primary composition, which can then be taken directly with water or by any other known means. This supplement may also include a sweetener, a stabilizer, an antioxidant, an additive, a flavoring or a colorant. A supplement for cosmetic purpose can additionally comprise a compound active with respect to the skin. Methods for preparing them are common knowledge.

In another embodiment, a pharmaceutical composition can be administered for prophylactic and/or therapeutic treatments. In therapeutic applications, compositions are administered to a patient already suffering from a disease, as described herein under, in an amount sufficient to cure or at least partially arrest the symptoms of the disease and its complications. An amount adequate to accomplish this is defined as “a therapeutically effective dose”. Amounts effective for this will depend on the severity of the disease and the weight and general state of the patient. In prophylactic applications, compositions according to the invention are administered to a patient susceptible to or otherwise at risk of a particular disease. Such an amount is defined to be “a prophylactic effective dose”. In this use, the precise amounts again depend on the patient's state of health and weight.

The primary composition of the invention is preferably administered with a pharmaceutically acceptable carrier, the nature of the carrier differing with the mode of administration, for example, enteral, oral and topical (including ophthalmic) putes. The desired formulation can be made using a variety of excipients including, for example, pharmaceutical grades of mannitol, lactose, starch, magnesium stearate, sodium saccharin, cellulose, magnesium carbonate. This composition may be a tablet, a capsule, a pill, a solution, a suspension, syrup, a dried oral supplement, a wet oral supplement.

It will be appreciated that the skilled person will, based on his own knowledge select the appropriate components and galenic form to target the active compound to the tissue of interest, e.g. the skin, colon, stomach, eyes, kidney or liver, taking into account the route of administration.

The invention also relates to a cosmetic composition comprising the primary composition described above. It may be formulated in lotions, shampoos, creams, sun-screens, after-sun creams, anti-ageing creams and/or ointments, for example. This composition which can be used topically additionally comprises a fat or oil which can be used in cosmetics, for example those mentioned in the CTFA work, Cosmetic Ingredients Handbook, Washington. It is also possible to add other cosmetically active ingredients. The composition additionally comprises a structuring agent and an emulsifier. Other excipients, colorants, fragrances or opacifiers can also be added to the composition. It will be appreciated that the present cosmetic products will contain a mixture of different ingredients known to the skilled person, ensuring a fast penetration of the said substance into the skin and preventing degradation thereof during storage.

It will be understood that the concept of the present invention may likewise be applied as an adjuvant therapy assisting in presently used medications. Since the compounds of the present invention may easily be administered together with food material special clinical food may be applied containing a high amount of the said substances. It will be clear that on reading the present specification together with the appending claims the skilled person will envisage a variety of different alternatives to the specific embodiments mentioned herein.

Administering to a pet or human, a food, nutritional supplement, a cosmetic or pharmaceutical composition as described above, results in an improved skin health, in particular for photo protection of the skin or for protecting skin tissue against ageing, e.g. for inhibiting damage to the skin and/or mucous membranes by inhibiting collagenases and enhancing the synthesis of collagen. In fact, the use of the primary composition as described above makes it possible to enhance the bioavailability of the said bioactive compounds in the body and to slow down the ageing of the skin, for example. It may also be useful in the prevention or treatment of sensible, dry or reactive skins, or for improving skin density or firmness.

The composition described here above may also be used for the preparation of an oral, topical or pharmaceutical composition for eyesight, in particular for reducing risk of cataract and age-related macular degeneration. It can be used also for preventing or treating cardiovascular diseases or disorders or cancers and for stimulating or improving the native immune system and reducing blood glucose, for example.

The following examples illustrate the invention in more detail without restricting the same thereto. All percentages are given by weight otherwise indicated.

### EXAMPLE 1

### Water-Dispersible Wolfberry Formulation

160 g of tap water and 2 g of sodium bicarbonate were introduced into a 0.5-liter glass-reactor. 40 g of dried wolfberry fruits containing 0.29% of zeaxanthin palmitate were added to the above solution, and the mixture was homogenized with a mixing device (Polytron®, at 26000 rpm) during 15 minutes. The temperature was maintained below 30° C. by cooling with an ice bath. The resulting mixture had a pH of 8.3. The mixture was then centrifuged at 2000 G for 10 minutes. The solid residue was discarded (50 g). The liquid phase (150 g) was neutralized to pH 7 by adding 0.8 g of 50% aqueous solution of citric acid. The red-orange liquid was lyophilized and 33.2 g of a red-orange powder was obtained.

This powder contains all the essential bioactive components of wolfberry and it can be dispersed in water to form a stable suspension. The zeaxanthin palmitate content of the powder is 0.28%; the yield in zeaxanthin palmitate is 80%.

### EXAMPLE 2

### Water-Dispersible Zeaxanthin Concentrate (2-Steps Process)

### 2.1. Removal of Water-Soluble Compounds (Mainly Sugars)

250 g of dried wolfberry fruits containing 0.29% of zeaxanthin palmitate were introduced into a 3.0-liter glass-reactor. 1.5 liter of tap water was added and the mixture was gently mixed at room temperature during 1 hour. The liquid phase was then separated and a second washing of the fruit solids was carried out with 1 liter of water under the same conditions. The two liquid phases were combined and lyophilized to obtain 157 g of powder that contains less than 0.02% of zeaxanthin palmitate.

### 2.2. Water-Dispersible Zeaxanthin Concentrate

The above water-washed wolfberry fruits were introduced into a 2-liter reactor equipped with a pH-electrode. Additional 250 g of tap water was added and the pH of the mixture was brought up to 10.5 by adding a 10% aqueous solution of sodium hydroxide. The mixture was homogenized with a mixing device (Polytron®, at 26000 rpm) during 15 minutes. During the homogenization the pH of the mixture was maintained at 10.5 by addition of a 10% aqueous solution of sodium hydroxide. The temperature was kept below 30° C. by cooling with an ice bath. The mixture was then centrifuged at 2000 G for 10 minutes. The solid residue was discarded. The liquid phase was pasteurized at 85° C. for 2 minutes and then neutralized to pH of 7.0 by adding 6.8 g of 50% aqueous solution of citric acid. The resulting liquid was lyophilized and 68.8 g of a red powder was obtained.

This powder can be dispersed in water to form a stable suspension. The zeaxanthin palmitate content of the powder is 0.81%. The yield in zeaxanthin palmitate is 76.8%.

### EXAMPLE 3

### Preparation of a Dairy Product

The primary composition as prepared in Example 1 is used for the manufacture of fermented yoghurt-like milk products. To do this, IL of a milk product containing 2.8% of fats and supplemented with 2% of skimmed milk powder and 6% of sucrose was prepared, pasteurized and its temperature then lowered to 42° C. Precultures of a non-thickening strain of Streptococcus thermophilus and of a non-viscous strain of Lactobacillus bulgaricus were reactivated in a sterile MSK culture medium containing 10% of reconstituted milk powder and 0.1% of commercial yeast extract. The pasteurized milk product is then inoculated with 1% of each of these reactivated precultures and this milk product was then allowed to ferment at 32° C. until the pH reached a value of 4.5. To the fermented milk, yoghurt-like product, the primary composition as in example 1 (1%) was added and stored at 4° C.

### EXAMPLE 4

### Preparation of a Pet Food Product

A feed mixture was made up of corn, corn gluten, chicken and fish, salts, vitamins and minerals. The moistened feed leaving the pre-conditioner was then fed into an extruder-cooker and gelatinised. The gelatinised matrix leaving the extruder was forced through a die and extruded. The extrudate leaving the die head was cut into pieces suitable for feeding to dogs, dried at about 110° C. for about 20 minutes, and cooled to form pellets. The resulting water activity of the pellets was about 0.6. The pellets were coated by spraying a coating substrate comprising tallow fat and the primary composition as prepared in Example 1.

### EXAMPLE 5

### Preparation of a Cosmetic for Oral Administration

A composition in the form of a hard capsule has the following formulation:

TABLE 1

| Compound | mg per capsule |
|---|---|
| primary composition of example 1 | 500 |
| Excipient for the core | |
| Microcrystalline cellulose | 70 |
| Encompress ™ | 60 |
| Magnesium stearate | 3 |
| Anhydrous colloidal silica | 1 |
| Coating agent | |
| Gum-lack | 5 |
| Talc | 61 |
| Sucrose | 250 |
| Polyvidone | 6 |
| Titanium dioxide | 0.3 |
| Colouring agent | 5 |

The composition can administered to the individual in an amount of 2 to 3 capsules daily.

1-19. (canceled)

20. A process for preparing a water dispersible primary composition of water extractible, lipophilic and non lipophilic bioactive components that are exclusively of fruit or vegetable or plant origin, which comprises:
a) subjecting a fruit, vegetable or plant material to homogenisation at a pH of 8 to 11 and at a temperature of 20 to 500° C. to form a homogenized mass,
b) separating a liquid extract from the homogenised mass and subsequently bringing the extract to pH neutrality to form a neutralized liquid extract, and
c) concentrating or drying the neutralized liquid extract to form the composition.

21. The process according to claim 20, wherein the selected fruit or vegetable or plant material is washed with water at room temperature prior to the homogenization.

22. The process according to claim 20, wherein the lipophilic bioactive components are lipids, carotenoids or lipophilic vitamins.

23. The process according to claim 20, wherein the non lipophilic bioactive components are alkaloids, carbohydrates, polyphenols, flavonoids, hydrosoluble vitamins or minerals.

24. The process according to claim 20, wherein step a) is performed on a crude or washed fruit or vegetable or plant material at a temperature lower than 300° C.

25. The process according to claim 20, wherein the drying according to step c) is performed by freeze drying.

26. A primary composition of water extractible, lipophilic and non-lipophilic bioactive components that are exclusively of fruit or vegetable or plant origin wherein the profile of the bioactive components is similar or identical to that of the corresponding whole fruit or vegetable.

27. The composition according to claim 26, wherein the lipophilic bioactive components are lipids, carotenoids or lipophilic vitamins.

28. The composition according to claim 27, wherein the carotenoids are lycopene, carotene, phytofluene, phytoene, canthaxanthin, astaxanthin, beta-cryptoxanthin, capsanthin, lutein, zeaxanthin or one of the preceding in the form of a fatty acid ester.

29. The composition according to claim 26, wherein the non-lipophilic bioactive components are alkaloids, carbohydrates, polyphenols, flavonoids, hydrosoluble vitamins or minerals.

30. The composition according to claim 29, wherein the non-lipophilic bioactive components are apigenin, luteolin, diosmetin or other flavones; quercetin, myricetin, kaempferol or other flavonols; naringenin, hesperidin, or other flavanones; epicatechin, gallocatechin, or other catechins; pelargonidin, malvidin, cyanidin or other anthocyanidins; or genistein, daidzein, or other isoflavones.

31. The composition according to claim 26, in the form of a water-dispersible powder composition.

32. The composition according to claim 26, wherein the fruit, vegetable or plant material is a wolfberry, blueberry, cranberry, white currant, red currant, blackcurrant, mulberry, blackberry, gooseberry, raspberry, sea buckthorn, strawberry, arbutus berry, grapes, or any other flavonoid, polyphenol or carotenoid-rich fruit; or vegetables, seeds, flowers or plant materials of apples, melons, kiwi, cherries, red date, prunes, peaches, persimmons, mandarin, orange, tangerine, grapefruit or other citrus fruits, chamomile, chrysanthemum, bitter orange, honeysuckle, jasmine, safflower, tomato, spinach, celery, carrots, pea, kale, parsley, watercress, cabbage, broccoli, lettuce, Brussels sprouts, collard greens, turnip greens, fennel, onions, tea, corn, cocoa, coffee, thyme or sweet red pepper.

33. A food product comprising the primary composition according to claim 26 for oral administration, in the form of a food supplement, a pet food product, a pet food supplement, a cosmetic preparation or a pharmaceutical preparation.

34. A food product for oral administration, a food supplement, a pet food product, a pet food supplement, a cosmetic preparation or a pharmaceutical preparation which comprises the primary composition made according to the process of claim 20.

35. A method for improving skin health which comprises preparing an oral, cosmetic or pharmaceutical composition from the composition according to claim 26 and administering a therapeutically effective amount of the composition to a subject's skin to provide protection of skin tissue against ageing.

36. A method for improving eye health which comprises preparing an oral, cosmetic or pharmaceutical composition from the composition according to claim 26 and administering a therapeutically effective amount of the composition to a subject's eye to provide eye health.

37. A method for stimulating a subject's immune system which comprises preparing an oral, cosmetic or pharmaceutical composition from the composition according to claim 26 and administering a therapeutically effective amount of the composition to a subject for stimulating the subject's immune system.

38. A method for treating cardiovascular diseases, cancers or diabetes which comprises preparing an oral, cosmetic or pharmaceutical composition from the composition according to claim 26 and administering a therapeutically effective amount of the composition to a subject for treating cardiovascular diseases or disorders or cancers or diabetes.
